(19) United States
(12) Patent Application Publication
(10) Pub. No.: US 2004/0259814 A1
(43) Pub. Date: Dec. 23, 2004

(54) Synthesis of solanum glycosides

(76) Inventor: Mohammed Shahid, Reading (GB)

Correspondence Address: FISH & NEAVE LLP, 1251 AVENUE OF THE AMERICAS, 50TH FLOOR, NEW YORK, NY, 10020-1105, US

(21) Appl. No.: 10/783,821

(22) Filed: Feb. 20, 2004

Related U.S. Application Data
(63) Continuation of application No. PCT/EP02/09349, filed on Aug. 21, 2002.

(30) Foreign Application Priority Data
Aug. 21, 2001 (EP) 01120144.9

(51) Int. Cl. C07J 17/00; A61K 31/704

(52) U.S. Cl. 514/26; 536/6.1

(57) ABSTRACT

The present invention relates to the chemical synthesis of solanum glycosides, in particular to the synthesis of solamargine as well as to novel β-monosaccharide Intermediate compounds.

The present invention relates to the chemical synthesis of solanum glycosides, in particular to the synthesis of solamargine as well as to novel β-monosaccharide intermediate compounds.

Solas dine and its glycosides are of considerable interest clinically. They are widely used as starting products for the synthesis of various steroidal drugs. Thus the aglycon solasodine is a source for synthetic cortisone and progesterone.

It is moreover well established that certain naturally occurring conjugate solasodine glycosides have potent antineoplastic properties. Of particular interest are the triglycosides solasonine (22R, 25R)-spiro-5-en-3β-yl-α-L-rhamnopyranosyl-(1->2 gal)-O-p-D-glucopyranosyl-(1->3 gal)-β-D-galactopyranose and solar-argine (22R, 25R)-spiro-5-en-3β-yl-α-L-rhamnopyranosyl-(1->2 glu)-α-L-rhamnopyranosyl-(1->4 glu)-β-D-gluco-pyranose. The structures of these triglycosides are shown below:

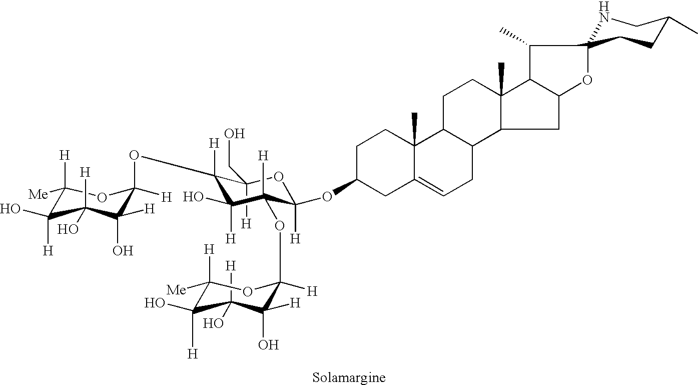

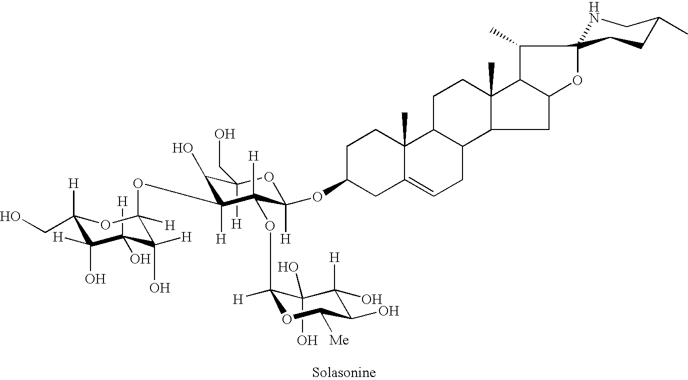

The above triglycosides are conventionally obtained by extraction from a plant source. Thus a commercially available extract of S. sodomaaeum, commonly referred to as BEC (Drug Future, 1988: vol. 13.8, pages 714-716) is a crude mixture of solamargine, solasonine and their isomeric diglycosides. The extraction process for making BEC involves homogenizing the fruits of S. sodomaeum in a large volume of acetic acid, filtering off the liquid through muslin followed by precipitation of the glycrosides with ammonia (Drugs of today (1990), Vol. 26 No. 1, p. 55-58, cancer letters (1991), Vol. 59, p. 183-192). The yield of the solasodine glycoside mixture is very low (approx. 1%). Moreover the individual process steps are not defined to GMP in terms of scale up, definition of yield composition and product quality.

There is thus a great need for a cost efficient process that provides the active antineoplastic triglycoside solamargine at high yield with little or no impurities.

Contrary to other steroid ring systems, the steroid skeleton of solasodine contains a very labile nitrogen containing ring. This aglycon can thus not readily be chemically modified while keeping the steroid skeleton intact. Thus, in spite of the fact that the aglycon solasodine is readily available, the prior art does not disclose the synthesis of the solamargine using the aglycon material as starting material.

The synthesis of solamargine requires the stereoselective glycosylation of solasodine at the relatively unreactive hydroxyl group.

It has been found that solasodine is not compatible with the conventional steroid glycosylation technique. Thus no glycosylation was observed following the treatment of solasodine with tetrabenzoyl α-D-glucopyranosyl trichloroacetimidate and trimethyl-silyl triflate or trifluoride etherate (unpublished results).

The problem underlying the present invention is to provide a cost effective method for the preparation of solamargine.

The present invention resides in the finding that the stereoselective β-glycosylation of solasodine may be achieved in high yields using specific glucopyranosyl donors. Preferably the reaction is carried out in the presence of a promoter.

### DETAILED DESCRIPTION OF THE INVENTION

Thus it was unexpectedly found that by reacting a D-glucopyranosyl donor of the following formula II

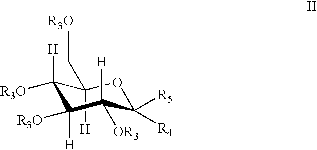

wherein each R₃ independently represents a benzoyl, acetyl or pivaloyl group,

wherein R₄ is halogen selected from Cl, Br or I and R₅ is hydrogen or

R₄ is hydrogen and R₅ is SEt or SPh,

with solasodine the correspondingly protected (3-glycoside of the formula I

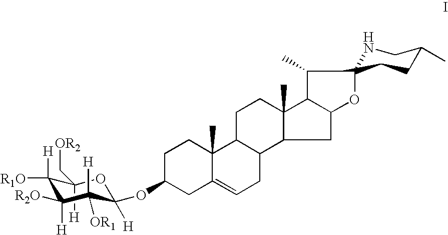

could be obtained in high yield.

As a D-glucopyranosyl donor tetra-O-bohzoyl-α-D-glucopyranoryl bromide is preferred.

Preferably the reaction is carried out in the presence of a promoter.

Any conventional promoter as used in saccharide chemistry may be used.

The following promoters are particularly preferred:

Silver triflate (silver trifluororhethane sulfonate), silver trifluoromethantriflate, boron trifluoride (−10° C.), diethyl etherate, trimethylsilyl triflate bromide, N-iodosuccinimide or dimethyl, thiomethyl sulfonium triflate, whereby silver triflate is most preferred.

The reaction is preferably carried out using dichloromethane as the solvent.

Preferably the reaction time is 30 min.-1 hr at −20° C.

The present invention also provides the following novel β-glycosides of formula I which may be used as intermediates for the synthesis of solamargine:

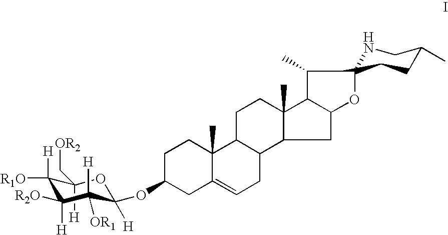

wherein each of R1 and R2, which may be the same or different, represent a conventional protecting group, preferably benzoyl, pivaloyl or acetyl.

The desired end product solamargine may be prepared by deprotecting the β-glycoside of formula I to obtain a compound of the formula V

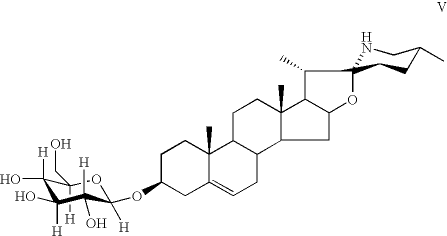

and optionally reesterifying the most reactive hydroxyl groups (OH-3 and OH-6) using conventional protecting groups to obtain a compound of formula IIa, wherein R2 is as defined above, whereby pivaloyl is preferred as a protection group,

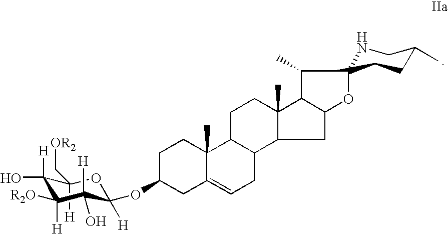

The partially protected β-glycoside diol is then glycosylated at OH-2 and OH-4 with a suitable α-L-rhamnopyranosyl donor.

Suitable rhamnose donors include tri-O-benzoyl-α-rhamnopyranosyl bromide, tri-O-pivaloyl-α-L-rhamnopyranosyl trichloroacetoimidate or a glyooside of formula IV

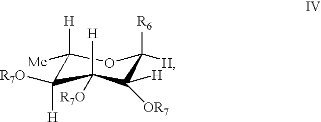

wherein R₆ is Br, Cl, I, SEt or SPh and

R₇ is any conventional protecting group, preferably benzoyl, acetyl or pivaloyl.

Tri-O-benzoyl-α-L-rhamnopyranosyl bromide is preferred as the rhamnose donor.

The protected solameargine of formula III (1) (2);

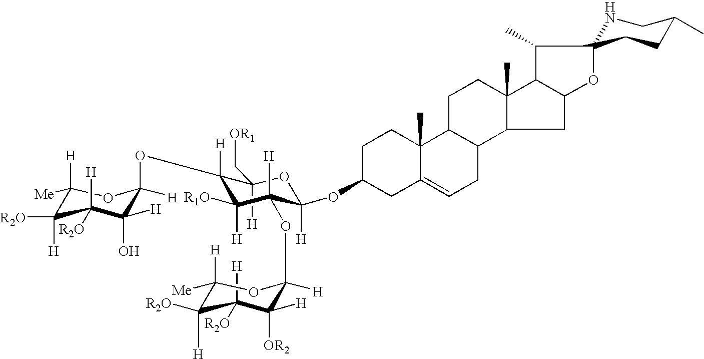

may be deesterified in a conventional manner, e.g. by treating the protected solamargine with a base such as sodium methoxide or sodium hydroxide in a methanol-dichloromethane solution or a methanol-tetrahydrofuran-water mixture, followed by neutralization with e.g. solid CO₂ or mild acid ion-exchange resin such as Arhberlyst® 50H⁺ or Dowex® (H+ form). These ion-exchange resins may also be used in any other deprotection step in the synthesis according to the invention.

The Examples described below serve only illustrative purposes and are not construed to limit the scope of the invention.

### SYNTHESIS EXAMPLE 1

Step A Preparation of bromo 2,3,4,6,-tetra-O-benzoyl-α-D-glucopyranose (tetra-O-benzoyl-α-D-glucopyranosyl bromide)

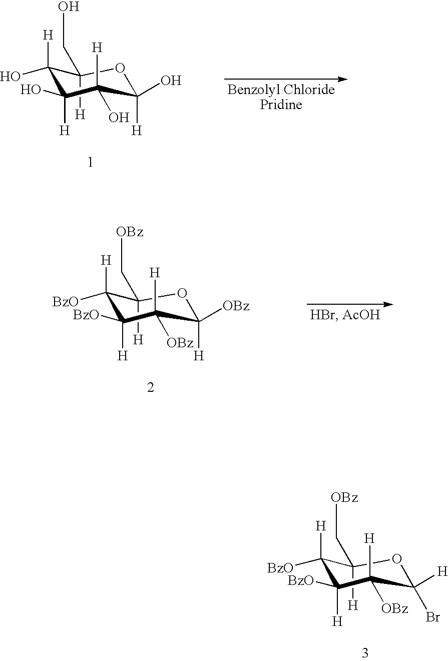

D-Glucose (30 g) (1) was placed in a 1 liter three necked round bottomed flask equipped with stirrer and thermometer. Pyridine (300 ml was added and the mixture gently heated to aid dissolution. The mixture was cooled to 10-12° C. using ice/water and benzoyl cloride (116 ml) added dropwise over a period of 40 min (temperature reached 20° C.). After about 90 ml of the benzoyl chloride had been added the mixture became more viscous and a light yellow precipitate formed. After the benzoyl chloride addition was complete, the mixture was left stirring overnight at room temperature when a light brown slurry formed. Water (400 ml) was added and the mixture extracted with dichloromethane (DCM) (3×800 ml). The organic phase was separated and washed with water (600 ml), 1N HCl (2×600 ml) and saturated NaHCO₃ (800 ml). The organic phase was dried (MgSO₄), filtered and the solvent removed to leave a thick oil (2).

The fully benzoylated glucose (2) was then dissolved in dichloromethane (200 ml) and cooled to 0° C. in a ice/water bath. Hydrogen bromide (32% in acetic acid, (142 ml)) was then added dropwise over 30 min to the reaction mixture. When addition was complete, the mixture was allowed to reach room temperature and stirred overnight. Dichloromethane (400 ml) (DCM) was added and the mixture washed with ice-water (4×500 ml), saturated NaHCO₃ (3×500 ml), dried with MgSO₄ and filtered through activated charcoal. The solvent was removed under reduced pressure to leave a light yellow oil which solidified on standing. The title compound (3) was recrystallised from diethylether (800 ml) and petroleum ether (700 ml) to give 85 g as an off white solid.

Step B Preparation of Bromo 2,3,4-tri-O-benzoyl-rhamnopyranose (tri-O-benzoyl-α-rhamnopyranosyl bromide)

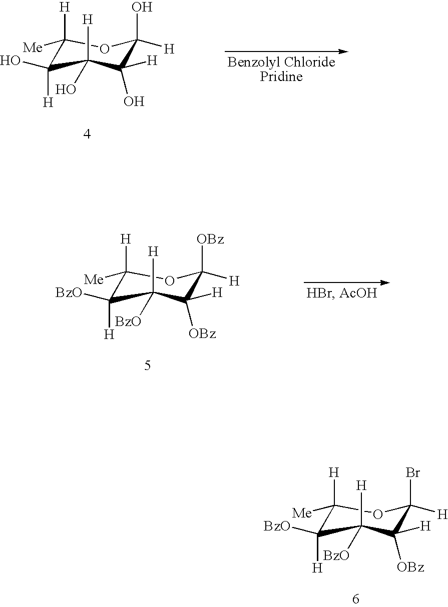

L-Rhamriose (20 g) (4) was placed in a 250 ml round bottomed flask equipped with stirrer, thermometer and pressure equalising dropping funnel. Pyridine (25 ml) was added and the mixture cooled to 0° C. using a ice/water bath. B enzoyl chloride (90 ml) was then added dropwise over 20 min, and after the addition was complete the mixture was heated at 60° C. for 2 h. After the mixture had cooled to room temperature, water (30 ml) was added, stirred for 20 min and then diluted with dichloromethane (DCM) (500 ml). The mixture was washed with cold water (2×200 ml), 1N HCl (3×25 ml), saturated NaHCd₃ (30⁶ ml) and saturated brine (300 ml). The organic phase was dried over MgSO₄, filtered through activated charcoal and the solvent removed under reduced pressure to give a thick syrup (5).

The fully benzoylated rhamnose (5) was dissolved in acetic acid (30 ml) and the solution cooled to 0° C. Hydrogen bromide (32% in acetic acid) was then added dropwise over 20 min. When addition was complete, the mixture was allowed to reach room temperature and was stirred overnight (18 h).

Dichloromethane (400 ml) was added and the mixture was washed with ice/water (2×200 ml), dried over MgSO₄, filtered and the solvent removed under reduced pressure to give an oil that crystallized on standing. Recrystallisation from toluene/petroleum ether provided the title compound (6) 23 g as a white solid.

Step C Preparation of solasodine-2,3,4,6-tetra-O-benzoyl-glucose (tetra-O-benzoyl-solasod-5en-3β-yl-D-glucopyranoside)

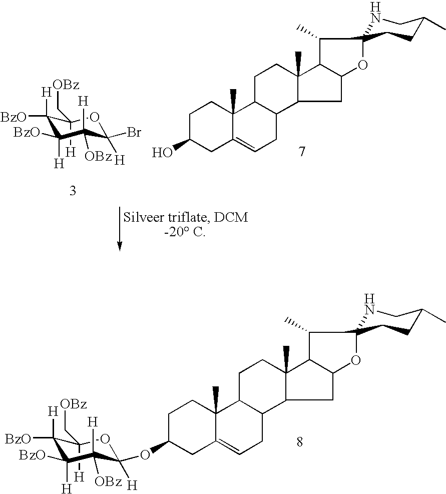

Solasodine (7) (15 g, 302 mmol), bromo-benzoyl-glucose (tetra-O-benzoyl-α-D-glucopyranosyl bromide) (27 g, 544 mmol) and 4 Å molecular sieve (crushed to a powder and preheated in a vacuum oven at 60° C.), were placed in a 560 ml round bottom flask equipped With nitrogen inlet-bubbler, pressure equalizing dropping funnel and low temperature thermometer. Anhydrous dichloromethane (250 ml) was then added and the mixture was stirred at room temperature under argon for 40 min. The mixture was then cooled to −20° c. A solution of silver trifluoromethane sulfonate (14 g, 544 mmol) in anhydrous toluene was then added dropwise over 15 min to the cold reaction mixture. After addition, the mixture was slowly allowed to warm to 5° C. yielding a viscous pale precipitate. Thin layer chromatography (TLC) of the reaction mixture (eluent; 10:90 MeOH: DCM) with solasodine (7) R_{f}=0.42, showed a spot at R_{f}=0.5 and excess bromo-glucose (3) at R_{f}=0.8. The reaction mixture was filtered through a pad of celite and washed with DCM (260 ml). The filtrate was then concentrated under reduced pressure to leave a thick dark brown oil. Silica gel chromatography (eluent; 40:60; EtOAc: toluene to 50:50 EtOAc: toluene) provided the pure product (8) (33.4 g, 100%) as a light orange crusty solid.

Step D Benzoyl Deprotection

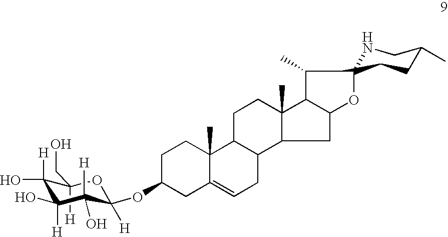

The fully protected solasodine-glucose adduct (8) (37 g, 377 mmol) was dissolved in methanol (400 ml) and dichloromethane (200 ml). To the homogenous mixture sodium Methoxide (12 ml, 25% by weight in methanol) was added and the reaction was mixture stirred at room temperature for 2 h. Thin layer chromatography (eluent: 10:90; MeOH:DCM) of the reaction mixture showed no remaining protected adduct (R_{f}=0.5, UV active) and only a higher running spot at R_{f}=0.9 (methylbenzoate), the product appeared at R_{f}=0.15 (UV inactive, visualized by H₂SO₄/MeOH charring). Amberlyst® 15H+ resin was activated by washing in 1N HCl, filtered and then added to the reaction mixture until the pH was between 7 and 8. The solvent was then removed under reduced pressure to give a thick brown syrup. Silica gel chromatography (eluent: 10:90; MeOH:DCM to 20.88:2: MeOH:DCM:NH₃ aq.) provided the pure deprotected product (9) (16.4 g, 76%).

Thin layer chromatography (20:88:2; MeOH:DCM:NH₃aq.) showed the product (at R_{f}=0.55 (UV inactive, with H₂SO₄/MeOH charring)

Step E Selective pivaloylation (3,6) of the glucose-solasodine adduct

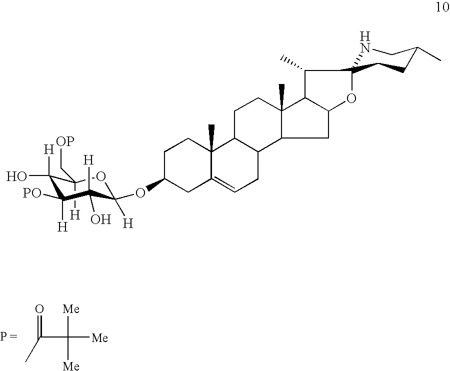

The glucose-solasodine adduct of step D (16 g, 278 mmol) (9) and pyridine (120 ml) were placed in a three necked flask equipped with a stirrer and a thermometer. The solution was cooled to 0° C. using ice/water and pivaloyl chloride (6.7 g, 556 mmol) was added dropwise over a period of 20 min. After about 30 min a TLC sample was divided between water and ethylacetate; the ethylacetate layer was used (eluent: 5:95; Me00H:DCM), which showed only the monopivaloyl product at R_{f}=0.2 and some of the starting material at R_{f}=0.1. Another 2 equivalents Of pivaloyl chloride (6.7 g) was added (TLC as before showed no starting material and only mono- and dipivaloyl products, at R_{f}=0.2 and 0.4, respectively). After another 90 min at 0° C. TLC showed only the dipivaloyl product and some minor impurities. The reaction mixture was diluted with ethylacetate and washed 3× with 5% HCl aq. (250 ml). The organic phase was washed with saturated brine, dried (MgSO₄), filtered and the solvent removed under reduced pressure to yield a brown oil. Silica gel chromatography (eluent: 5:95; MeOH:DCM to 10:90 MeOH:DCM) provided the product (10) as an off white solid (15 g, 72%).

Step F Addition of two moles of rhamnose to the 3,6-protected glucose-solasodine adduct

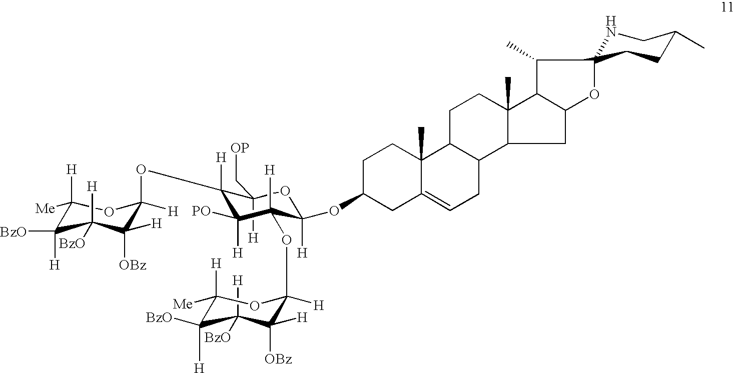

The rhamnose bromide from step B (6)(19 g, 355 mmol), dipivaloyl-glucosesolasodine (12 g, 161 mmol), 4 Å molecular sieves (30 g, preheated in an oven at 50° C.) were placed in a three necked flask equipped with a stirrer, a low temperature thermometer, an argon inlet-bubbler and a pressure dropping equalizing funnel. Anhydrous DOCM was added and the mixture was stirred at room temperature for 40 min under argon.

The mixture was then cooled to −30° C. and a solution of silver triflate in toluene (80 ml) was added dropwise over 20 min maintaining the temperature at −30° C. After addition the resulting light yellow precipitate was stirred at −20° C. for 30 min and then slowly allowed to reach −5° C. over 35 min. A TLC sample (eluent: 5:95; MeOH:DCM) showed a UV active product at R_{f}=0.4 and no starting material at R_{f}=0.3. 3 ml triethylamine was added and the mixture was diluted with DCM and filtered through a pad of celite. The celite was washed with DCM (200 ml) and the filtrate was evaporated under reduced pressure to leave a purple coloured syrup. Silica gel chromatography (5:95 to 10:90; MeOH:DCM) provided the pure fully protected solamargine (11) (13,6 g, 50%) as a crustly light yellow solid.

Step G Deprotection of Fully Protected Solamargine

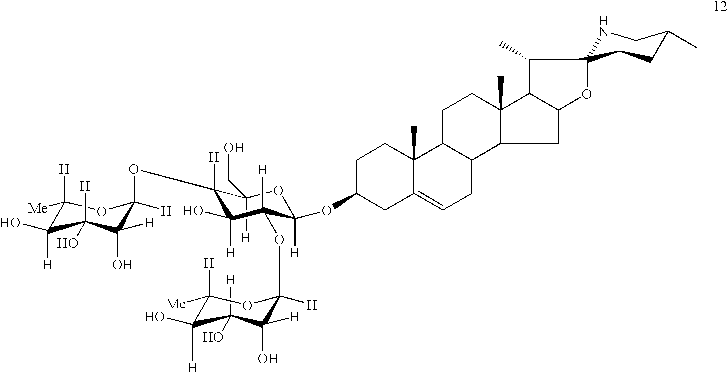

A mixture of TH° F., MEOH and water (1:1:1; 20:20:20 ml) was added to the fully protected solamargine (11) (8 g, 4.8 mmol). Sodium hydroxide (1.9 g, 48 mmol) was then added and the resulting reaction mixture was heated gently at 40° C. for 18 h. The mixture was cooled and diluted with methanol (50 ml) and neutralized with Amberlyst 50H+ until the pH was between 7 and 8. The mixture was filtered and the filtrate was evaporated under reduced pressure to give a thick brown semi-solid mass. TLC showed a product at R_{f}=0.45 (eluent: 70:28:2; DCM:MeOH:NH₃ aq.), which was identical to authentic solamargine.

Silica gel chromatography (eluent: 80:20; DCM:MeOH to 78:20:2 DCM:MeOH:NH₃aq) gave a product (3.4 g) at R_{f}=0.45 with a close running spot at R_{f}=0.47.

Further Purification:

The semi solid paste was taken up in 3% acetic acid (450 ml) and stirred for 20 min until nearly all dissolved. The solution was decanted from any undissolved material and adjusted with concentrated ammonia to pH 8 at which point a fine precipitate formed. The precipitate was subjected to high speed centrifugation and the supernatant decanted from the pellet that had formed. The pellet was then washed three times with water and then dispersed in water (100 ml) and freeze dried to give a light white solid (1.52 g). The 78% pure solamargine (12) was then further purified with conventional reversed phase HPLC chromatography.

The obtained product solamargine [(22R, 25R)-spiro-5-en-3β-yl-α-L-rhamnopyranosyl-(1-2glu)-O-α-L-rhamnopyranosyl-(1-4glu)-β-D-glucopyranose] was subjected further analysis.

A sample of the final compound was analysed by positive ion Electrospray—Mass Spectroscopy using a PE Sciex API 150 EX single quadrupole mass spectrometer. A major signal was observed at m/z 868.7 which is consistent With the expected protonated monoisotopic mass of solamargine.

Moreover the HPLC retention time of synthetic Solamargine was identical to authentic natural material, as was the thin layer chromatography (TLC).

The NMR and mass spectra were consistent with the expected structure.

### SYNTHESIS EXAMPLE 2

Step C Preparation of solasodine-2,3,4,6-tetra-O-benzoyl-glucose (tetra-O-benzoyl-solasod-5en-3β-yl-D-glucopyranoside)

Solasodine (7) (3.6 g from Research Plus Inc, USA) and tetra-O-benzoyl-α-D-glucopyranosyl bromide (3) (8.60 g) in dichloromethane (120 ml) was stirred with powdered molecular sieve (4 Å) for 50 min. with cooling to −12° C. A solution of silver triflate (3.35 g) in toluene (30 ml) was added dropwise over 20 min at −12 to −10° C. The mixture was stirred for a further 30 min with slow warming to −5° C. The mixture was then filtered through Celite and washed with dichloromethane. The filtrate was washed with brine, saturated aqueous sodium bicarbonate (twice), again brine, dried, filtered and concentrated onto silica gel. The material was then chromatographed on silica eluting with EtOAc: toluene (40:60) and the combined fractions concentrated to a white solid (8). The yield was 7.13 g and the sample was crystallised by trituration with ethanol. The NMR and mass spectra were consistent with the expected structure.

Step D Benzoyl Deprotection

A solution of tetra-O-benzyl-solasod-5en-3β-yl-β-D-glucopyranoside (8) (1.0 g) and sodium methoxide (0.2 g) in dichloromethane (80 ml), and methanol was stirred overnight at 50° C. The resulting mixture was allowed to cool and neutralized with Dowex-50 (H+ form), filtered and the solvent removed under reduced pressure to leave a solid residue which was finally purified by silica chromatography to obtain the pure deprotected material (9).

The NMR and mass spectra were consistent with the expected structure.

1. A glucose-sclaeodine conjugate of the general formula I or a derivative thereof 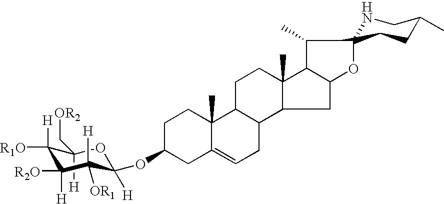
wherein each of R₁ and R₂ are the same- or different and
represents, ¹ is a benzoyl or a pivaloyl group.

2. A method for the preparation of the glucose-solasocline conjugate as defined in claim 1, comprising the reaction of solasodine with a gluoopyranoryl donor of general formula II 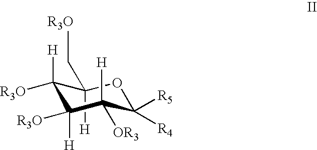
wherein each R₃ independently represents a benzoyl, acetyl or pivaloyl group,
wherein R₄ is halogen selected from Cl, Br or I and R₅ is hydrogen or
R₄ is hydrogen and R₅ is SEt or SPh,
followed by optionally de-protecting the obtained glycoside to yield a compound of the formula V 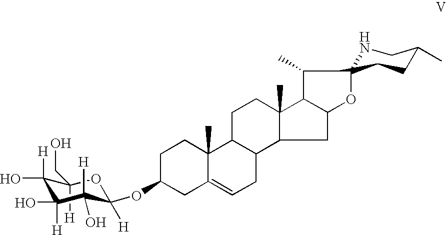
and reestentication of the most reactive hydroxyl groups (OH-3 and OH-6) to yield a compound of the formula IIa 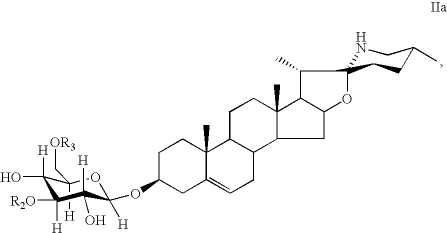
wherein R₂ is a group selected from pivaloyl or acetyl.

3. A method for the preparation of solamargine comprising the glycogylation of the diol of formula IIa 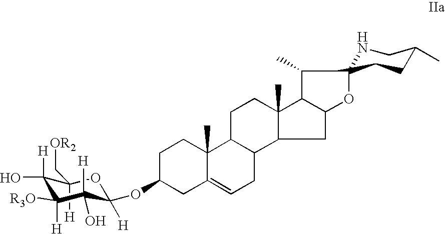
wherein R₂ is defined as in claim 1 with an α-L-rhamnopyranosyl donor to yield protected solamargine of formula III (1) which is de-esterified to yield solamargine of formula III (2)

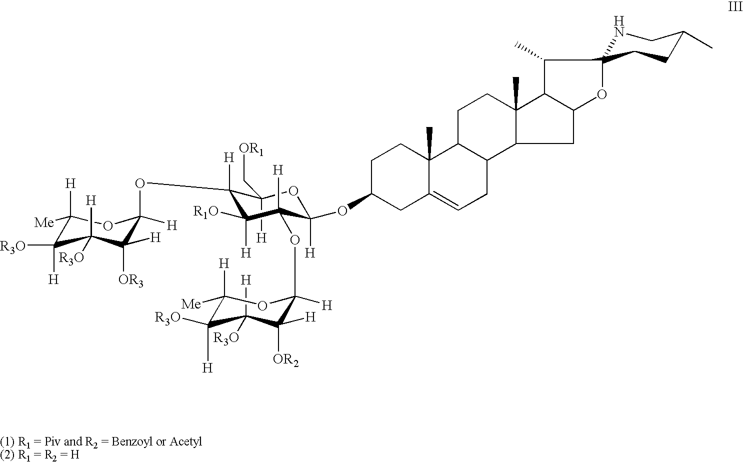

4. The method according to claim 2, wherein the D-glucosepyranosyl donor is tetra-O-benzoy-α-D-glucopyranosyl bromide, tetra-O-acetyl-α-D-glucopyranosyl bromide or tetra-O-pivaloy-α-D-glucopyranosyl bromide.

5. The method according to claim 2 or 4, wherein the glycosylation reaction is carried out in the presence of a promoter selected from silver trifluoromethane sultonate (sliver triflate), boron trifluoride diothyl etherate, trimethysilyl triflate bromide, N-iodosuccinimide or dimethyl thiomethyl sulfonium triflate, silver trifluoromethyltriflate.

6. The method of claim 2, wherein the protected glycoside is deprotected in methanol-dichloromethane solution by treatment with sodium methoxide, followed by neutralization with solid CO₂ or mild acid ion-exchange resin.

7. The method of claim 2, wherein the most reactive hydroxyl groups (OH-3 and OH-6) are protected by reesterification with pivaloyl chloride in pyridine solution.

8. The method of claim 3, wherein the rhamnose donor is tri-O-benzoyl-α-L-rhamnopyranosyl bromide, tri-O-plvaloyl-α-L-rhamnopyranosyl trichloroacetimidate or a glyooside of the general formula IV 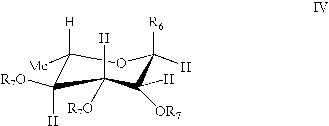
wherein R₆ is Br, Cl, I, SEt or SPh and
R₇ is benzoyl, acetyl or pivaloyl.

9. The method of claim 3, wherein the protected solamargine is de-esterified by, treatment with a base selected from sodium methoxide or sodium hydroxide in methanol-dichloromethane solution or a methanol-tetrahydrofuran-water mixture followed by neutralization with solid CO₂ or mild acid ion-exchange resin.
